(19) United States
(12) Patent Application Publication
(10) Pub. No.: US 2002/0165240 A1
(43) Pub. Date: Nov. 7, 2002

(54) Method of treating proliferative diseases using Eg5 inhibitors

(76) Inventors: Spencer David Kimball, East Windsor, NJ (US); Louis J. Lombardo, Belle Mead, NJ (US); David B. Rawlins, Morrisville, PA (US); Hai-Yun Xiao, Princeton, NJ (US); Deborah L. Roussell, Trenton, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/108,403

(22) Filed: Mar. 28, 2002

Related U.S. Application Data
(60) Provisional application No. 60/279,956, filed on Mar. 29, 2001.
(60) Provisional application No. 60/280,366, filed on Mar. 30, 2001.

(51) Int. Cl. A61K 31/519; A61K 31/44; A61K 31/513

(52) U.S. Cl. 514/258.1; 514/269; 514/344

(57) ABSTRACT

The invention provides a method for treating a condition via modulation of the Eg5 protein activity comprising administering to a mammalian species in need of such treatment an effective amount of at least one small molecule Eg5 protein inhibitor. The invention also provides a method for treating a condition via modulation of the Eg5 protein activity comprising administering to a mammalian species in need of such treatment an effective amount of at least one small molecule Eg5 protein inhibitor in combination with at least one other anti-cancer agent.

### RELATED APPLICATIONS

This application claims the benefit under 35 U.S.C. Section 119(e) of U.S.

Provisional Patent Application No. 60/279,956 filed Mar. 29, 2001, and Provisional Patent Application No. 60/280,366 filed Mar. 30, 2001.

### FIELD OF INVENTION

This invention relates to methods of treating proliferative diseases, such cancer, using an inhibitor of the kinesin-like Eg5 motor protein and to methods of treating cancer using an Eg5 inhibitor in combination with other antineoplastic agents.

### BACKGROUND

The maintenance of cell populations within an organism is governed by the cellular processes of cell division and programmed cell death. Within normal cells, the cellular events associated with the initiation and completion of each process is highly regulated. In proliferative disease such as cancer, one or both of these processes may be perturbed. For example, a cancer cell may have lost its regulation (checkpoint control) of the cell division cycle through either the overexpression of a positive regulator or the loss of a negative regulator, perhaps by mutation.

Alternatively, a cancer cell may have lost the ability to undergo programmed cell death through the overexpression of a negative regulator. Hence, there is a need to develop new chemotherapeutic drugs that will restore the processes of checkpoint control and programmed cell death to cancerous cells.

One approach to the treatment of human cancers is to target a protein that is essential for cell cycle progression. In order for the cell cycle to proceed from one phase to the next, certain prerequisite events must be completed. There are checkpoints within the cell cycle that enforce the proper order of events and phases. One such checkpoint is the spindle checkpoint that occurs during the metaphase stage of mitosis. Small molecules that target proteins with essential functions in mitosis may initiate the spindle checkpoint to arrest cells in mitosis. Of the small molecules that arrest cells in mitosis, those which display anti-tumor activity in the clinic also induce apoptosis, the morphological changes associated with programmed cell death. An effective chemotherapeutic for the treatment of cancer may thus be one which induces checkpoint control and programmed cell death. Unfortunately, there are few compounds available for controlling these processes within the cell. Most compounds known to cause mitotic arrest and apoptosis act as tubulin binding agents. These compounds alter the dynamic instability of microtubules and indirectly alter the function/structure of the mitotic spindle thereby causing mitotic arrest. Because most of these compounds specifically target the tubulin protein which is a component of all microtubules, they may also affect one or more of the numerous normal cellular processes in which microtubules have a role. Hence, there is also a need for small molecules that more specifically target proteins associated with proliferating cells.

Eg5 is one of several kinesin-like motor proteins that are localized to the mitotic spindle and known to be required for formation and/or function of the bipolar mitotic spindle. Recently, there was a report of a small molecule that disturbs bipolarity of the mitotic spindle (Mayer, T. U. et. al. 1999. Science 286(5441) 971-4, herein incorporated by reference). More specifically, the small molecule induced the formation of an aberrant mitotic spindle wherein a monoastral array of microtubules emanated from a central pair of centrosomes, with chromosomes attached to the distal ends of the microtubules. The small molecule was dubbed “monastrol” after the monoastral array. This monoastral array phenotype had been previously observed in mitotic cells that were immunodepleted of the Eg5 motor protein. This distinctive monoastral array phenotype facilitated identification of monastrol as a potential inhibitor of Eg5. Indeed, monastrol was further shown to inhibit the Eg5 motor-driven motility of microtubules in an in vitro assay. The Eg5 inhibitor monastrol had no apparent effect upon the related kinesin motor or upon the motor(s) responsible for golgi apparatus movement within the cell. Cells that display the monoastral array phenotype either through immunodepletion of Eg5 or monastrol inhibition of Eg5 arrest in M-phase of the cell cycle. However, the mitotic arrest induced by either immunodepletion or inhibition of Eg5 is transient (Kapoor, T. M., 2000. J Cell Biol 150(5) 975-80). Both the monoastral array phenotype and the cell cycle arrest in mitosis induced by monastrol are reversible. Cells recover to form a normal bipolar mitotic spindle, to complete mitosis and to proceed through the cell cycle and normal cell proliferation. These data suggest that a small molecule inhibitor of Eg5 which induced a transient mitotic arrest may not be effective for the treatment of cancer cell proliferation. Nonetheless, the discovery that monastrol causes mitotic arrest is intriguing and hence there is a need to further study and identify compounds which can be used to modulate the Eg5 motor protein in a manner that would be effective in the treatment of human cancers. There is also a need to explore the use of these compounds in combination with other antineoplastic agents.

Another recent report proposes that retinoic acid interferes with the cell cycle and delays progression through G2/M phase by modulation of Eg5 gene expression (Kaiser, A., et. al., 1999. J Biol Chem 274(27), 18925-31, herein incorporated by reference). Like the mitotic arrest induced by the immunodepletion of Eg5 protein and by monastrol's inhibition of Eg5, the mitotic arrest induced by retinoic acid is transient.

It is, therefore, an object of the present invention to provide a method for the treatment of proliferative diseases, such as cancer, using an Eg5 inhibitor. Additionally, it is an object of the present invention to provide a method for the treatment of cancer using a combination that consists of an Eg5 inhibitor and other antineoplastic agents. These and other objects of the present invention will become more apparent from the description thereof set forth below.

### SUMMARY

The present invention provides a method for treating a condition via modulation of Eg5 protein activity comprising administering to a mammalian species in need of such treatment an effective amount of at least one small molecule Eg5 protein inhibitor. The invention also provides a method for treating a condition via modulation of Eg5 protein activity comprising administering to a mammalian species in need of such treatment an effective amount of at least one small molecule Eg5 protein inhibitor in combination with at least one other anti-cancer agent.

### DESCRIPTION

Listed below are definitions of various terms used to describe the compounds used in the methods of the instant invention. These definitions apply to the terms as they are used throughout the specification (unless they are otherwise limited in specific instances) either individually or as part of a larger group.

The term “alkyl” herein alone or as part of another group refers to a monovalent alkane (hydrocarbon) derived radical containing from 1 to 12 carbon atoms unless otherwise defined. An alkyl group is an optionally substituted straight, branched or cyclic saturated hydrocarbon group. When substituted, alkyl groups may be substituted with up to four substituent groups, R as defined, at any available point of attachment. When the alkyl group is said to be substituted with an alkyl group, this is used interchangeably with “branched alkyl group”. Exemplary unsubstituted such groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl, and the like. Exemplary substituents may include but are not limited to one or more of the following groups: halo (such as F, Cl, Br, I), haloalkyl (such as CCl₃ or CF₃), alkoxy, alkylthio, hydroxy, carboxy (—COOH), alkyloxycarbonyl (—C(O)R), alkylcarbonyloxy (—OCOR), amino (—NH₂), carbamoyl (—NHCOOR— or —OCONHR—), urea (—NHCONHR—) or thiol (—SH). Alkyl groups as defined may also comprise one or more carbon to carbon double bonds or one or more carbon to carbon triple bonds.

The term “alkenyl” herein alone or as part of another group refers to a hydrocarbon radical straight, branched or cyclic containing from 2 to 12 carbon atoms and at least one carbon to carbon double bond.

The term “alkynyl” herein alone or as part of another group refers to a hydrocarbon radical straight, branched or cyclic containing from 2 to 12 carbon atoms and at least one carbon to carbon triple bond.

The numbers in the subscript after the symbol “C” define the number of carbon atoms a particular group can contain. For example “C₁₋₆ alkyl” means a straight or branched saturated carbon chain having from one to six carbon atoms; examples include methyl, ethyl, n-propyl, isopropyl, n-butyl, sec-butyl, isobutyl, t-butyl, n-pentyl, sec-pentyl, isopentyl, and n-hexyl. Depending on the context, “C₁₋₆ alkyl” can also refer to C₁₋₆ alkylene which bridges two groups; examples include propane-1,3-diyl, butane-1,4-diyl, 2-methyl-butane-1,4-diyl, etc. “C₂₋₆ alkenyl” means a straight or branched carbon chain having at least one carbon-carbon double bond, and having from two to six carbon atoms; examples include ethenyl, propenyl, isopropenyl, butenyl, isobutenyl, pentenyl, and hexenyl. Depending on the context, “C₂₋₆ alkenyl” can also refer to C₂₋₆ alkenediyl which bridges two groups; examples include ethylene-1,2-diyl (vinylene), 2-methyl-2-butene-1,4-diyl, 2-hexene-1,6-diyl, etc. “C₂₋₆ alkynyl” means a straight or branched carbon chain having at least one carbon-carbon triple bond, and from two to six carbon atoms; examples include ethynyl, propynyl, butynyl, and hexynyl.

The term “cycloalkyl” herein alone or as part of another group is a specie of alkyl containing from 3 to 15 carbon atoms, without alternating or resonating double bonds between carbon atoms. It may contain from 1 to 4 rings. Exemplary unsubstituted such groups include cyclopropyl, cyclobutyl, cyclopentyl, cyclohexyl, adamantyl, etc. Exemplary substituents include one or more of the following groups: halogen, alkyl, alkoxy, alkyl hydroxy, amino, nitro, cyano, thiol and/or alkylthio.

The terms “alkoxy” or “alkylthio” herein alone or as part of another group denote an alkyl group as described above bonded through an oxygen linkage (—O—) or a sulfur linkage (—S—), respectively.

The term “alkyloxycarbonyl” herein alone or as part of another group denotes an alkoxy group bonded through a carbonyl group. An alkoxycarbonyl radical is represented by the formula: —C(O)OR, where the R group is a straight or branched C₁₋₆ alkyl group.

The term “alkylcarbonyl” herein alone or as part of another group refers to an alkyl group bonded through a carbonyl group.

The term “alkylcarbonyloxy” herein alone or as part of another group denotes an alkylcarbonyl group which is bonded through an oxygen linkage.

The term “arylalkyl” herein alone or as part of another group denotes an aromatic ring bonded to an alkyl group as described above.

The term “aryl” herein alone or as part of another group refers to monocyclic or bicyclic aromatic rings, e.g. phenyl, substituted phenyl and the like, as well as groups which are fused, e.g., napthyl, phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. Aryl groups may optionally be substituted with one or more groups including, but not limited to halogen, alkyl, alkoxy, hydroxy, carboxy, carbamoyl, alkyloxycarbonyl, nitro, trifluoromethyl, amino, cycloalkyl, cyano, alkyl S(O)m (m=O, 1, 2), or thiol.

The term “carbocyclic ring” herein alone or as part of another group refers to stable, saturated or partially unsaturated monocyclic ring hydrocarbyls of 3 to 7 carbon atoms such as cyclopropyl, cyclobutyl, cyclopentyl, cyclohexyl and cycloheptyl. The carbocyclic ring may be optionally substituted meaning that the carbocyclic ring may be substituted at one or more substitutable ring positions by one or more groups independently selected from alkyl (preferably lower alkyl), alkoxy (preferably lower alkoxy), nitro, monoalkylamino (preferably a lower alkylamino), dialkylamino (preferably a di[lower]alkylamino), cyano, halo, haloalkyl (preferably trifluoromethyl), alkanoyl, aminocarbonyl, monoalkylaminocarbonyl, dialkylaminocarbonyl, alkyl amido (preferably lower alkyl amido), alkoxyalkyl (preferably a lower alkoxy[lower]alkyl), alkoxycarbonyl (preferably a lower alkoxycarbonyl), alkylcarbonyloxy (preferably a lower alkylcarbonyloxy) and aryl (preferably phenyl), said aryl being optionally substituted by halo, lower alkyl and lower alkoxy groups.

The term “cycloalkyl” herein alone or as part of another group refers to fully saturated and partially unsaturated hydrocarbon rings of 3 to 9, preferably 3 to 7 carbon atoms. Further, a cycloalkyl may be substituted. A substituted cycloalkyl refers to such rings having one, two, or three substituents, preferably one, selected from the group consisting of halo, alkyl, substituted alkyl, alkenyl, alkynyl, nitro, cyano, oxo (═O), hydroxy, alkoxy, thioalkyl, —CO₂H, —C(═O)H, CO₂—alkyl, —C(═O)alkyl, keto, ═N—OH, ═N—O-alkyl, aryl, heteroaryl, heterocyclo, a five or six membered ketal (i.e. 1,3-dioxolane or 1,3-dioxane), —NR′R″, —C(═O)NR′R″, —CO₂NR′R″, —C(═O)NR′R″, —NR′CO₂′R″, —NR′C(═O)R″, —SO₂NR′R″, and —NR′SO₂′R″, wherein each of R′ and R″ is independently selected from hydrogen, alkyl, substituted alkyl, and cycloalkyl, or R′ and R″ together form a heterocyclo or heteroaryl ring.

The term “heteroaryl” herein alone or as part of another group refers to substituted and unsubstituted aromatic 5 or 6 membered monocyclic groups, 9 or 10 membered bicyclic groups, and 11 to 14 membered tricyclic groups which have at least one heteroatom (O, S or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quatemized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. The heteroaryl ring system may contain zero, one, two or three substituents selected from the group consisting of halo, alkyl, substituted alkyl, alkenyl, alkynyl, nitro, cyano, hydroxy, alkoxy, thioalkyl, —CO₂H, —C(═O)H, —CO₂-alkyl, —C(═O)alkyl, phenyl, benzyl, phenylethyl, phenyloxy, phenylthio, cycloalkyl, substituted cycloalkyl, heterocyclo, heteroaryl, —NR′R″, —C(═O)NR′R″, —CO₂NR′R″, —C(═O)NR′R″, —NR′CO₂′R″, —NR′C(═O)R″, —SO₂NR′R″, and —NR′SO₂′R″, wherein each of R′ and R″ is independently selected from hydrogen, alkyl, substituted alkyl, and cycloalkyl, or R′ and R″ together form a heterocyclo or heteroaryl ring.

Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, triazinyl and the like.

Exemplary bicyclic heteroaryl groups include indolyl, benzothiazolyl, benzodioxolyl, benzoxaxolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridinyl, dihydroisoindolyl, tetrahydroquinolinyl and the like.

Exemplary tricyclic heteroaryl groups include carbazolyl, benzidolyl, phenanthrollinyl, acridinyl, phenanthridinyl, xanthenyl and the like.

The term “heterocycloalkyl” herein alone or as part of another group refers to a cycloalkyl group (nonaromatic) in which one of the carbon atoms in the ring is replaced by a heteroatom selected from O, S or N, and in which up to three additional carbon atoms may be replaced by said heteroatoms.

The term “heterocyclic ring” herein alone or as part of another group refers to a stable, saturated, or partially unsaturated monocyclic ring system containing 5 to 7 ring members of carbon atoms and other atoms selected from nitrogen, sulfur and/or oxygen. Preferably, a heterocyclyl is a 5 or 6-membered monocyclic ring and contains one, two, or three heteroatoms selected from nitrogen, oxygen and/or sulfur. The heterocyclic ring may be optionally substituted which means that the heterocyclic ring may be substituted at one or more substitutable ring positions by one or more groups independently selected from alkyl (preferably lower alkyl), alkoxy (preferably lower alkoxy), nitro, monoalkylamino (preferably a lower alkylamino), dialkylamino (preferably a di[lower]alkylamino), cyano, halo, haloalkyl (preferably trifluoromethyl), alkanoyl, aminocarbonyl, monoalkylaminocarbonyl, dialkylaminocarbonyl, alkyl amido (preferably lower alkyl amido), alkoxyalkyl (preferably a lower alkoxy[lower]alkyl), alkoxycarbonyl (preferably a lower alkoxycarbonyl), alkylcarbonyloxy (preferably a lower alkylcarbonyloxy) and aryl (preferably phenyl), said aryl being optionally substituted by halo, lower alkyl and lower alkoxy groups. Examples of such heterocyclic rings are isoxazolyl, imidazolinyl, thiazolinyl, imidazolidinyl, pyrrolyl, pyrrolinyl, pyranyl, pyrazinyl, piperidyl, morpholinyl and triazolyl. The heterocyclic ring may be attached to the parent structure through a carbon atom or through any heteroatom of the heterocyclyl that results in a stable structure.

The term “heterocyclyl” herein alone or as part of another group as used herein refers to a stable, saturated, or partially unsaturated, monocyclic, bridged monocyclic, bicyclic, and spiro ring system containing carbon atoms and other atoms selected from nitrogen, sulfur and/or oxygen. Preferably, a heterocyclyl is a 5 or 6-membered monocyclic ring or an 8-11 membered bicyclic ring which consists of carbon atoms and contains one, two, or three heteroatoms selected from nitrogen, oxygen and/or sulfur. The term “optionally substituted” as it refers to “heterocyclyl” herein indicates that the heterocyclyl group may be substituted at one or more substitutable ring positions by one or more groups independently selected from alkyl (preferably lower alkyl), alkoxy (preferably lower alkoxy), nitro, monoalkylamino (preferably a lower alkylamino), dialkylamino (preferably a di[lower]alkylamino), cyano, halo, haloalkyl (preferably trifluoromethyl), alkanoyl, aminocarbonyl, monoalkylaminocarbonyl, dialkylaminocarbonyl, alkyl amido (preferably lower alkyl amido), alkoxyalkyl (preferably a lower alkoxy[lower]alkyl), alkoxycarbonyl (preferably a lower alkoxycarbonyl), alkylcarbonyloxy (preferably a lower alkylcarbonyloxy) and aryl (preferably phenyl), said aryl being optionally substituted by halo, lower alkyl and lower alkoxy groups. Examples of such heterocyclyl groups are isoxazolyl, imidazolinyl, thiazolinyl, imidazolidinyl, pyrrolyl, pyrrolinyl, pyranyl, pyrazinyl, piperidyl, morpholinyl and triazolyl. The heterocyclyl group may be attached to the parent structure through a carbon atom or through any heteroatom of the heterocyclyl that results in a stable structure.

The term “heteroatom” means O, S or N, selected on an independent basis. It should be noted that any heteroatom with unsatisfied valences is assumed to have the hydrogen atom to satisfy the valences.

The term “halogen” or “halo” refers to chlorine, bromine, fluorine or iodine selected on an independent basis.

The term “amino” herein alone or as part of another group refers to —NH₂. An “amino” may optionally be substituted with one or two substituents, which may be the same or different, such as alkyl, aryl, arylalkyl, alkenyl, alkynyl, heteroaryl, heteroarylalkyl, cycloheteroalkyl, cycloheteroalkylalkyl, cycloalkyl, cycloalkylalkyl, haloalkyl, hydroxyalkyl, alkoxyalkyl, thioalkyl. carbonyl or carboxyl. These substituents may be further substituted with a carboxylic acid, any of the alkyl or aryl substituents set out herein. In some embodiments, the amino groups are substituted with carboxyl or carbonyl to form N-acyl or N-carbamoyl derivatives When a functional group is termed “protected”, this means that the group is in modified form to preclude undesired side reactions at the protected site. Suitable protecting groups for the compounds of the present invention will be recognized from the present application taking into account the level of skill in the art, and with reference to standard textbooks, such as Greene, T. W. et al., Protective Groups in Organic Synthesis, Wiley, N.Y. (1991).

As used herein, the phrase “radiation therapy” includes, but is not limited to, x-rays or gamma rays which are delivered from either an externally applied source such as a beam or by implantation of small radioactive sources.

As used herein, the phrase “antineoplastic agent” refers to compounds which prevent cancer cells from multiplying. In general, the antineoplastic agents of this invention prevent cancer cells from multiplying by: (1) interfering with the cell's ability to replicate DNA, or (2) inducing apoptosis in the cancerous cells.

As used herein, the term “patient” encompasses all mammalian species.

Suitable examples of salts of the compounds used in the methods of the invention with inorganic or organic acids are hydrochloride, hydrobromide, sulfate, methanesulfonate, maleate, fumarate, and phosphate. Salts which are unsuitable for pharmaceutical uses but which can be employed, for example, for the isolation or purification of free compounds I or their pharmaceutically acceptable salts, are also included.

All stereoisomers of the compounds of the instant invention are contemplated, either in admixture or in pure or substantially pure form. The definition of the compounds according to the invention embraces all possible stereoisomers and their mixtures. It very particularly embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates by conventional methods, such as, for example, salt formation with an optically active acid followed by crystallization.

It should be understood that the present invention includes prodrug forms of the compounds of formula I. Various forms of prodrugs are well known in the art. For examples of such prodrug derivatives, see:

(a) Design of Prodrugs, edited by H. Bundgaard (Elsevier, 1985); and Methods in Enzymology, Vol. 42, pp. 309-396, edited by K. Widder et al., (Academic Press, 1985);

(b) A Textbook of Drug Design and Development, edited by Krosgaard-Larsen and H. Bundgaard, Chapter 5, “Design and Application of Prodrugs,” by H. Bundgaard, pp. 113-191 (1991);

(c) H. Bundgaard, Advanced Drug Deliver Reviews, 8, pp. 1-38 (1992);

(d) H. Bundgaard et al., Journal of Pharmaceutical Sciences, 77, 285 (1988); and

(e) N. Kayeka et al., Chem. Phar. Bull., 32, 692 (1984).

The present invention relates to a method of treating a condition via modulation of the Eg5 protein activity comprising administering to a mammalian species in need of such treatment an effective amount at least one small molecule Eg5 protein inhibitor. The invention also provides a method for treating a condition via modulation of the Eg5 protein activity comprising administering to a mammalian species in need of such treatment a combination (simultaneous or sequential) of at least one antineoplastic agent and at least one small molecule Eg5 protein inhibitor. In a preferred embodiment, the condition treated is a proliferative disease such as cancer. Any compounds that act as antineoplastic agents and any small molecule which modulates the Eg5 protein sufficiently to induce mitotic arrest and apoptosis can be used in the instant invention. Monastrol has not been shown to induce apoptosis and is not included within the scope of this invention. In addition, the instant invention does not include antisense oligonucleotides designed from the HsEg5 gene sequence.

The small molecule Eg5 motor protein inhibitor can be any compound, such as those described in the U.S. Patent Application that was filed on Mar. 22, 2002 with the attorney docket number LD 0300, entitled “Cyano-substituted Dihydropyrimidine Compounds and their Use to Treat Diseases” (serial number to be determined), the disclosure of which is herein incorporated by reference, or pharmaceutically acceptable salts thereof, that has shown efficacy in treating cancer through the induction of mitotic arrest and apoptosis, or the potential to treat cancer through the induction of mitotic arrest and apoptosis. Preferred compounds used in the methods of the instant invention include compounds having formulae I, IIA, or IIIA, shown below.

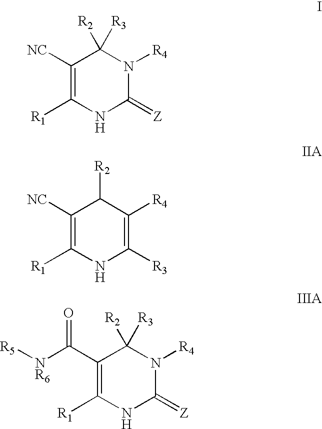

their enantiomers, diastereomers, pharmaceutically acceptable salts, prodrugs and solvates thereof wherein R₁ is hydrogen, alkyl or cycloalkyl; R₂ and R₃ are each independently H, alkyl, aryl, heteroaryl, arylalkyl, cycloalkylalkyl, heterocycloalkylalkyl or heteroarylalkyl. Alternatively, R₂ and R₃ may be taken together to form a either a carbocyclic or heterocyclic ring. R₄ is alkyl, arylalkyl, cycloalkylalkyl, heteroarylalkyl, heterocycloalkylalkyl, CN, COR₅, CO₂R₅ or CONR₅R₆. R₅ and R₆ are each independently H, alkyl, arylalkyl, cycloalkylalkyl, heteroarylalkyl or heterocycloalkylalkyl. Z is O, S or NR₈; R₈ is H, CN, sulfonamido, OR₇, alkyl, cycloalkyl, aryl, arylalkyl, heterocyclyl, heteroaryl or heteroarylalkyl. R₇ is H, alkyl, arylalkyl, cycloalkylalkyl, heterocycloalkylalkyl, or heteroarylalkyl.

In a preferred embodiment, the small molecules used in the methods of the instant invention comprise compounds of formula I or IIA

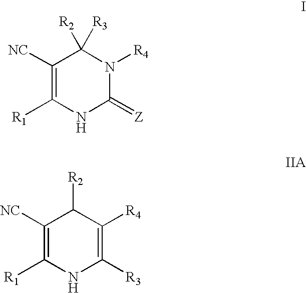

One preferred embodiment of the instant invention are compounds of formula I or IIA wherein R₁ is alkyl; R₂ is selected from the group consisting of aryl and heteroaryl; R₃ is H; R₄ is selected from the group consisting of alkyl, arylalkyl, CO₂R₅, and CONR₅R₆; R₅ and R₆ are independently selected from the group consisting of H, alkyl and arylalkyl; Z is selected from the group consisting of O, S, and NR₈; and R₈ is selected from the group consisting of H and CN.

In another preferred embodiment, the invention comprises compounds of formula I or IIA as defined above, wherein R₄ is selected from the group consisting of alkyl, arylalkyl, CO₂R₅, and CONR₅R₆.

In yet another preferred embodiment, the instant invention comprises the compounds of formula I or IIA, as defined above, wherein R₄ is CO₂R₅ or CONR₅R₆ and Z is O.

In yet a further preferred embodiment, the instant invention comprises the compounds of formula I or IIA, as defined above, wherein R₄ is selected from the group consisting of alkyl and arylalkyl, and Z is O.

In still yet another preferred embodiment, the instant invention comprises the compounds of formula I or IIA, as defined above, wherein R₁ is CH₃; R₂ is aryl; R₄ is CO₂R₅; R₅ is alkyl; and Z is O.

In still yet another preferred embodiment, the instant invention comprises the compounds of formula I or IIA, as defined above, wherein R₁ is CH₃; R₂ is aryl; R₄ is CONR₅R₆; R₅ is alkyl, R₆ is H; and Z is O.

When the invention employs combination (administered together or sequentially) therapy, the small molecule Eg5 protein inhibitor may be used with known anti-cancer treatments such as radiation therapy or with cytostatic or cytotoxic agents, such as for example, but not limited to, DNA interactive agents, such as cisplatin or doxorubicin; topoisomerase II inhibitors, such as etoposide; topoisomerase I inhibitors such as CPT-11 or topotecan; tubulin interacting agents, such as paclitaxel, docetaxel or the epothilones; hormonal agents, such as tamoxifen or Casodex; thymidilate synthase inhibitors, such as 5-fluorouracil; inhibitors of famesyltransferase, such as BMS-214662; inhibitors of cyclin dependent kinases such as flavopiridol, and anti-metabolites, such as methoxtrexate.

Furthermore, combination therapy may include the small molecule Eg5 inhibitor formulated in a fixed dose with the other anti-cancer agent(s). If formulated as a fixed dose, such combination products employ the compounds of this invention within the effective dosage range and the other pharmaceutically active agent or treatment within its approved dosage range. Compounds used in the methods of the instant invention may also be administered sequentially with known anticancer or cytotoxic agents when a combination formulation is inappropriate. The invention is not limited in the sequence of administration; small molecule Eg5 protein inhibitor(s) may be administered either prior to or after administration of the known anticancer or cytotoxic agent(s).

When combination therapy is employed, it is anticipated that the therapeutic effect of the instant invention may be achieved with smaller amounts of the antineoplastic agents and Eg5 protein inhibitors than would be required if such antineoplastic agents and Eg5 inhibitors were administered alone, thereby avoiding or minimizing adverse toxicity effects.

As discussed in the background section, Eg5 is a kinesin-like motor protein that facilitates spindle bipolarity during mitosis of the cell cycle. More specifically, the Eg5 protein may act to sort and bundle microtubules of the mitotic spindle during mitosis. Accordingly, Eg5 participates in cell cycle regulation through the spindle checkpoint during the M phase of the cycle. While not wishing to be bound by any theory, it is believed that the compounds used in the methods of the instant invention act as Eg5 inhibitors. The compounds use in the methods of the instant invention are contemplated to also inhibit other motor proteins, for example, including but not limited to: those human motor proteins that correspond to, Xklp2, MKLP1, CHO1, chromokinesins, Nod, Cenp-E, and MCAK, members of the BimC family, and members of the Kar3 family. Thus, the invention also provides a method for treating a condition, including proliferative diseases such as cancer, via modulation of motor proteins that correspond to: Xklp2, MKLP1, CHO1, chromokinesins, Nod, Cenp-E, and MCAK, members of the BimC family, and members of the Kar3 family comprising administering to mammalian species in need of such therapy an effective amount of at least one small molecule inhibitor of said motor proteins and may optionally may be used in combination with other anti-cancer agents. Additionally, compounds used in the methods of the instant invention are also envisioned to act as inhibitors of other kinesin or kinesin-like proteins and thus be effective in the treatment of diseases associated with other kinesin or kinesin-like proteins. Hence the invention further provides a method for treating a condition, including proliferative diseases such as cancer, via modulation of kinesin or kinesin-like protein(s) comprising administering to a mammalian species in need of such therapy an effective amount of at least one small molecule kinesin or kinesin-like protein inhibitor and may optionally may be used in combination with other anti-cancer agents.

The compound(s) used in the methods of the invention causes disruption of the bipolar spindle, initiates the spindle checkpoint, induces mitotic arrest, induces programmed cell death and inhibits tumor cell proliferation.

In contrast to agents such as retinoic acid and monastrol which have been reported to induce a transient arrest in mitosis through the modulation of Eg5 expression or activity, respectively, the small molecule(s) used in the methods of the instant invention, through inhibition of Eg5 motor protein activity, induces a cell cycle arrest in mitosis that is not transient but rather which progresses into programmed cell death. Furthermore, compounds used in the instant invention exhibit high potency, induce mitotic arrest and apoptosis in human cells in vitro, preferrable compounds do so at concentrations at or less than about 10 μM.

In contrast to agents such as retinoic acid which exert pleiotrophic effects upon cells, the small molecule compounds of the instant invention do not directly modulate the gene expression of numerous regulatory genes.

In contrast to microtubule agents, the instant invention does not disrupt the dynamic instability of microtubules. The instant invention, through inhibition of the Eg5 motor protein, may therefore more specifically target the mitotic spindle of proliferating cells, which may provide for different toxicity profiles than those of existing anti-cancer drugs.

Certain compounds of the present invention may generally be prepared according to the following schemes and the knowledge of one skilled in the art. Solvates (e.g., hydrates) of the compounds of the instant invention are also within the scope of the present invention. Methods of solvation are generally known in the art. Accordingly, the compounds of the instant invention may be in the free or hydrate form, and may be obtained by methods exemplified by the following schemes below.

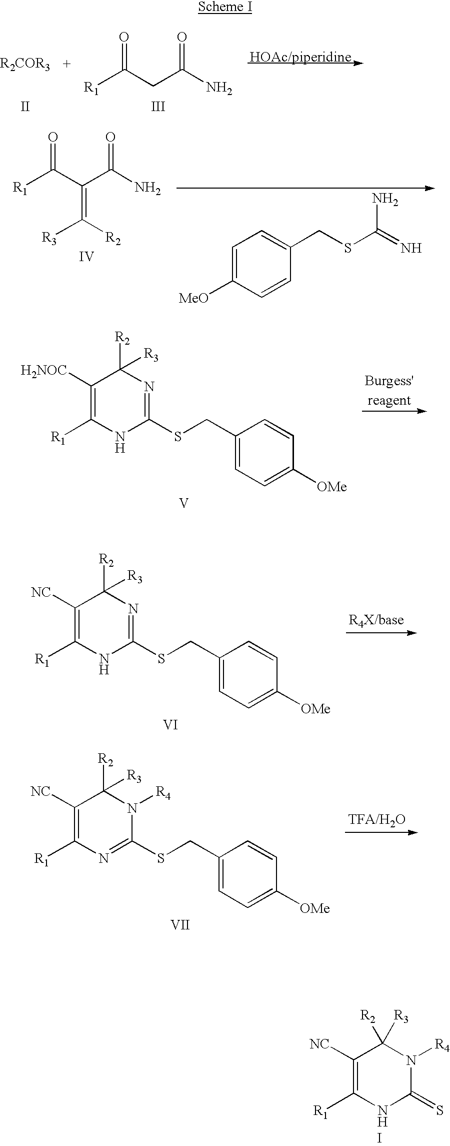

Compounds of formula I where Z is S may be made in accordance with Scheme I. A ketone or an aldehyde I (e.g., benzaldehyde, where R₂ is phenyl and R₃ is H), is condensed with an acetoacetamide III to give a Knoevenagel product IV as a mixture of isomers. Reaction with S-paramethoxybenzyl thiourea provides the protected dihydropyrimidine thione V. The primary amido group of V is dehydrated to the cyano substituent in VI using a dehydrating agent such as Burgess' reagent (methoxycarbonylsulfamoyl)triethylammonium hydroxide, inner salt. The N3 substituent is introduced by reaction with R₄X where R₄ is alkyl or acyl, and X is a leaving group, or where R₄X is an isocyanate or haloformate. The protecting group is removed by treatment with acid in the presence of water to give compounds of formula I where Z is S.

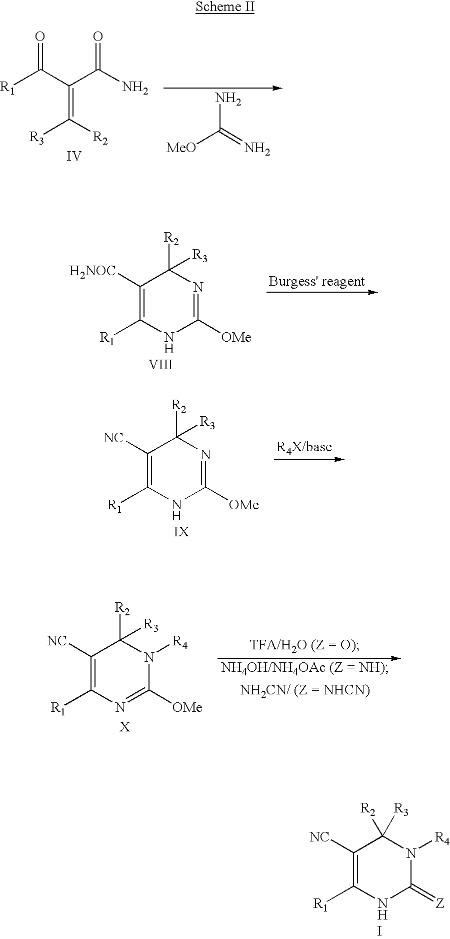

Compounds of formula I where Z is O, NH, or NR₈ are prepared from the reaction of Knoevenagel products IV with O-methyl isourea to provide the O-methyl dihydropyrimidines VIII. The primary amide is converted to a nitrile group using a dehydrating agent such as Burgess' reagent. The N3 substituent is introduced by reaction with R₄X where R₄ is alkyl or acyl, and X is a leaving group, or where R₄X is an isocyanate or haloformate. The methyl ether protecting group is removed by treatment with acid in the presence of water to give compounds of formula I where Z is O. Alternatively, treatment of compounds of formula X with ammonium hydroxide in the presence of ammonium acetate, or cyanamide in ethanol, provides compounds of formula I where Z is NH or NR₈.

Compounds of formula I may also be prepared using the Bignelli reaction (D. J. Brown in The Pyrimidines, Wiley: New York, 1962, 440).

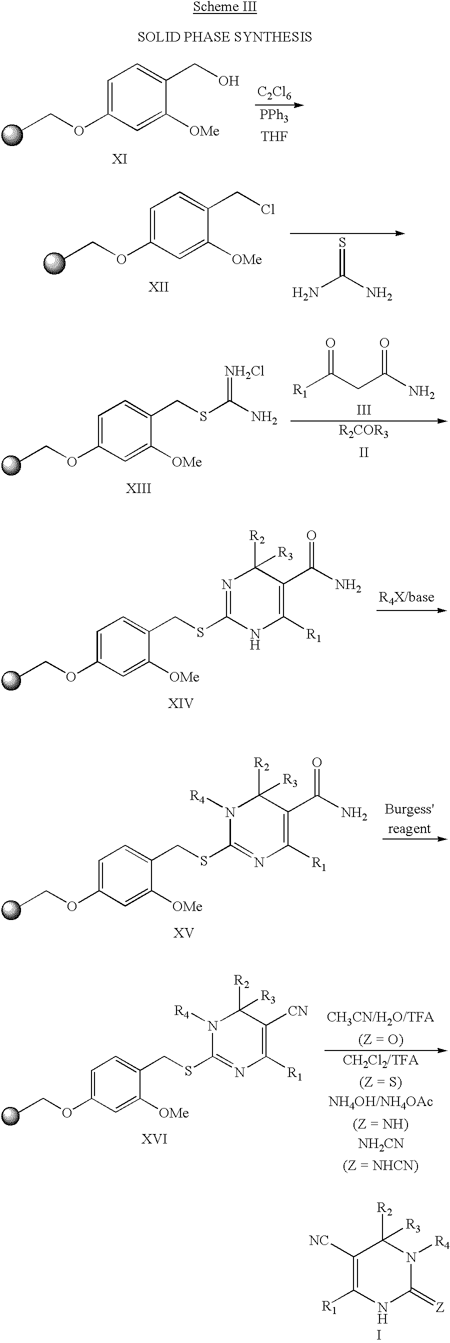

Compounds of formula I could be prepared on solid support as outlined in Scheme III. Starting compound XI denotes a resin-bound benzyl alcohol used for solid support synthesis which is prepared from a Merrifield resin denoted as [symbol] , and 2-methoxy-4-hydroxybenzaldehyde, followed by reduction of the aldehyde with reducing agents such as NaBH₄. The benzyl alcohol is converted into the benzyl chloride using agents such as hexachloroethane and triphenylphosphine in THF to form resins of formula XII. The chloride is displaced with thiourea to form the isothiourea resin XIII. The resulting resin is treated with excess of ketoamides like acetoamide (III, R₁ is CH₃), in the presence of ketones of formula R₂COR₃ or aldehydes of formula R₂CHO to form the resin-bound pyrimidinethiones of formula XIV. The N3 substituent is introduced using R₄X, where X is a leaving group and R₄ is alkyl or acyl, or R₄X is an isocyanate, or haloformate, in the presence of base to form structures of formula XV. The primary amide can be dehydrated to the cyano group using reagents such as Burgess' reagent to form compounds of formula XVI.

The products can be cleaved from the resin using a variety of conditions to form compounds of formula I, where Z is determined by the cleavage method employed.

Cleavage in the presence of aqueous acid will form compounds of formula I with Z being O, whereas cleavage under anhydrous acid conditions will form compounds of formula I with Z being S. Alternatively, treatment of resins with structure XVI with ammonium hydroxide in the presence of ammonium acetate will form compounds of formula I with Z being NH, while treatment with cyanamide, provides compounds of formula I with Z being NHCN.

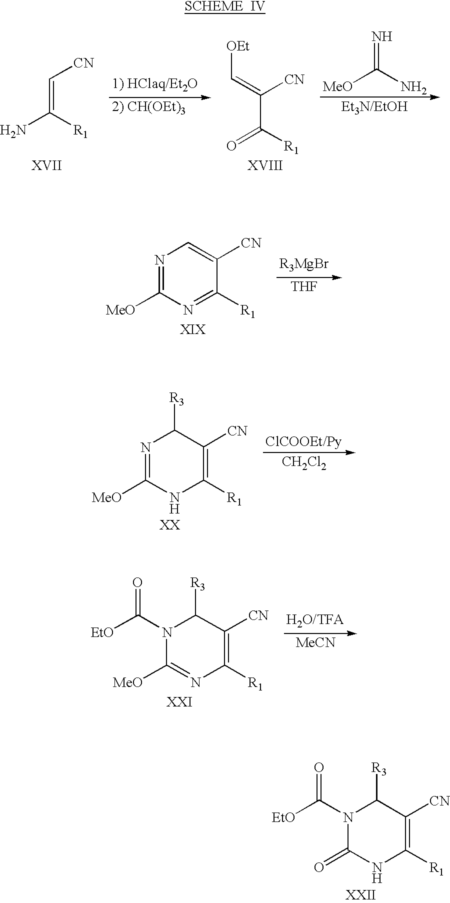

Compounds of formula XVM may be prepared from a 3-amino-3-alkyl acrylonitrile XVII using the methods illustrated in Scheme IV. Reaction of a compound of formula XVII with aqueous acid, such as hydrochloric acid, followed by treatment with triethyl orthoformate, provides a compound of formula XVIII. Reaction of a compound of formula XVIII with O-methyl isourea in the presence of a base such as triethylamine, provides a pyrimidine of formula XIX. Pyrimidines of formula XIX may be reacted with organometallic species such as a Grignard reagent, R₃MgBr, in a solvent such as ether or tetrahydrofuran, to give a pyrimidine of formula XX, which is a compound of formula IX wherein R₂ is H. In analogy with Scheme II, a compound of formula XX may be converted into a compound of formula XXII, which is a compound of formula I in which R4 is ethoxycarbonyl and R₂ is H.

In all of the above schema, a 2-acyl acetonitrile derivative, i.e., R₁COCH₂CN, may be substituted for a compound of formula III.

Dihydropyridine analogues of formula IIA may be prepared following the general process described in Scheme V.

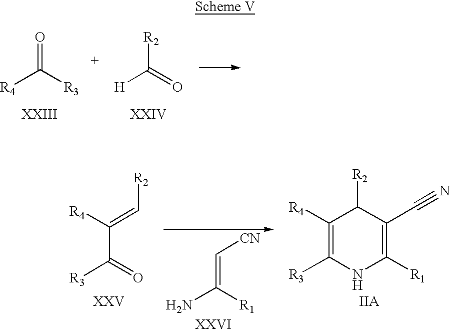

Thus, suitable dihydropyridine derivatives of formula IIA can be synthesized by the condensation of acetates of structure XXIII with aldehydes of structure XXIV using either acetic acid and pyridine with azetropic removal of water (Chem. Pharn. Bull 1992, 40, 2423-31) or bismuth trichloride (Chem. Lett. 1992, 10, 1945-6) to form the compounds of structure XXV. These compounds (XXV) undergo condensation with 3-aminocrotononitrile (XXVI) upon heating in ethanol to produce the dihydropyridines of structure IIA in high yield.

More specifically, a compound having formula IIA (XXVII) can be obtained by Scheme VI:

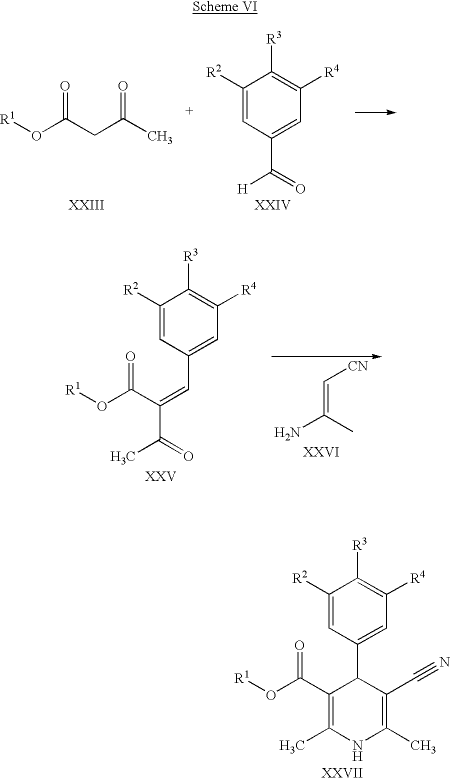

The condensation of acetoacetates of structure XXIII with aldehydes of structure XXIV using either acetic acid and pyridine with azetropic removal of water (Chem. Pharm. Bull. 1992, 40, 2423-31) or bismuth trichloride (Chem. Lett. 1992, 10, 1945-6) to form the benzylidene compounds of structure XXV. These benzylidenes (XXV) undergo condensation with 3-aminocrotononitrile (XXVI) upon heating in ethanol to produce the dihydropyridines of structure XXVII in high yield.

The compounds according to the invention have pharmacological properties; in particular, the small molecules used in the methods of the instant invention induce mitotic arrest and are believed to be Eg5 inhibitors. The novel compounds of the instant invention are thus useful in the therapy of a variety of proliferative diseases (including but not limited to diseases that could be treated via modulation of the Eg5 motor protein activity) such as cancer, autoimmune diseases, viral diseases, fungal diseases, neurodegenerative disorders and cardiovascular disease.

The present invention provides methods for the treatment of a variety of cancers, including, but not limited to, the following:

1. carcinoma including that of the bladder, breast, colon, kidney, liver, lung (including small cell lung cancer), ovary, prostate, testes, pancreas, esophagus, stomach, gall bladder, cervix, thyroid, and skin (including squamous cell carcinoma);

2. hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, and Burketts lymphoma;

3. hematopoietic tumors of myeloid lineage including acute and chronic myelogenous leukemias, myelodysplastic syndrome, and promyelocytic leukemia;

4. tumors of the central and peripheral nervous system including astrocytoma, neuroblastoma, glioma, and schwannomas;

5. tumors of mesenchymal origin including fibrosarcoma, rhabdomyoscarcoma, and osteosarcoma; and

6. other tumors including melanoma, xenoderma pigmentosum, keratoactanthoma, seminoma, thyroid follicular cancer, and teratocarcinoma.

In a preferred embodiment of this invention, the method of the present invention is used for the treatment of cancerous tumors. Advantageously, the method of this invention reduces the development of tumors, reduces tumor burden, or produces tumor regression in a mammalian host.

Antineoplastic agents which are suitable for use in the methods and compositions of this invention include, but are not limited to, microtuble-stabilizing agents such as paclitaxel (also known as Taxol®), docetaxel (also known as Taxotere®), 7-O-methylthiomethylpaclitaxel (disclosed in U.S. Pat. No. 5,646,176, herein incorporated by reference), 3′-tert-butyl-3′-N-tert-butyloxycarbonyl-4-deacetyl-3′-dephenyl-3′-N-debenzoyl-4-O-methoxycarbonyl-paclitaxel (disclosed co-pending U.S. Application Serial No. 60/179,965 filed on Feb. 3, 2000, herein incorporated by reference), C-4 methyl carbonate paclitaxel (disclosed in WO 94/14787, herein incorporated by reference), epothilone A, epothilone B, epothilone C, epothilone D, desoxyepothilone A, desoxyepothilone B, [1S-[1R*, 3R*(E), 7R*, 10S*, 11R*, 12R*, 16S*]]-7-11-dihydroxy-8,8,10,12,16-pentamethyl-3-[1-methyl-2-(2-methyl-4-thiazolyl)ethenyl]-4-aza-17-oxabicyclo[14.1.0]heptadecane-5,9-dione (disclosed in WO 99/02514, herein incorporated by reference), [1S-[1R*, 3R*(E), 7R*, 10S*, 11R*, 12R*, 16S*]]-3-[2-[2-(aminomethyl)-4-thiazolyl]-1-methylethenyl]-7,11-dihydroxy-8,8,10,12,16-pentamethyl-4-17-dioxabicyclo[14.1.0]-heptadecane-5,9-dione (disclosed in co-pending U.S. application Ser. No. 09/506,481 filed on Feb. 17, 2000, herein incorporated by reference), and derivatives thereof; microtuble-disruptor agents; inhibitors of cyclin dependent kinases (including those disclosed in U.S. Pat. No. 6,040,321, herein incorporated by reference); inhibitors of farnesyltransferase; alkylating agents; anti-metabolites; epidophyllotoxin; an antineoplastic enzyme; a topoisomerase inhibitor; procarbazine; mitoxantrone; platinum coordination complexes; biological response modifiers; growth factor inhibitors; hormonal/antihormonal therapeutic agents; and haematopoietic growth factors.

Classes of antineoplastic agents suitable for use in the present invention include, but are not limited to, the anthracycline family of drugs, the vinca drugs, the mitomycins, the bleomycins, the cytotoxic nucleosides, the taxanes, the epothilones, discodermolide, the pteridine family of drugs, diynenes, aromatase inhibitors, and the podophyllotoxins. Particularly useful members of those classes include, for example, paclitaxel, docetaxel, 7-O-methylthiomethylpacliitaxel, 3′-tert-butyl-3′-N-tert-butyloxycarbonyl-4-deacetyl-3′-dephenyl-3′-N-debenzoyl-4-O-methoxycarbonyl-paclitaxel, C-4 methyl carbonate paclitaxel, epothilone A, epothilone B, epothilone C, epothilone D, desoxyepothilone A, desoxyepothilone B, [1S-[1R*, 3R*(E), 7R*, 10S*, 11R*, 12R*, 16S*]]-7,11-dihydroxy-8,8,10,12,16-pentamethyl-3-[1-methyl-2-(2-methyl-4-thiazolyl)ethenyl]-4-aza-17-oxabicyclo[14.1.0]heptadecane-5,9-dione, [1S-[1R*, 3R*(E), 7R*, 10S*, 11R*, 12R*, 16S *]]-3-[2-[2-(aminomethyl)-4-thiazolyl]-1-methylethenyl]-7,11-dihydroxy-8,8,10,12,1 6-pentamethyl-4,17-dioxabicyclo[14.1.0]-heptadecane-5,9-dione, doxorubicin, carminomycin, daunorubicin, aminopterin, methotrexate, methopterin, dichloro-methotrexate, mitomycin C, porfiromycin, 5-fluorouriacil, 6-mercaptopurine, gemcitabine, cytosine arabinoside, podophyllotoxin or podophyllotoxin derivatives such as etoposide, etoposide phosphate or teniposide, melphalan, vinblastine, vincristine, leurosidine, vindesine, leurosine, and the like. Other useful antineoplastic agents include estramustine, cisplatin, carboplatin, cyclophosphamide, bleomycin, tamoxifen, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons, interleukins, and inhibitors of cyclin dependent kinases including, but not limited to, those in U.S. Pat. No. 6,040,321, herein incorporated by reference; and inhibitors of farnesyltransferase including, but not limited to, those in U.S. Pat. No. 6,011,029 herein incorporated by reference.

Preferred classes of antineoplastic agents are the taxanes and the epothilones, and the preferred antineoplastic agents are paclitaxel, docetaxel, 7-O-methylthio-methylpaclitaxel, 3′-tert-butyl-3′-N-tert-butyloxycarbonyl-4-deacetyl-3′-dephenyl-3′-N-debenzoyl-4-O-methoxycarbonyl-paclitaxel, C-4 methyl carbonate paclitaxel, epothilone A, epothilone B, epothilone C, epothilone D, desoxyepothilone A, desoxyepothilone B, [1S-[1R*, 3R*(E), 7R*, 10S*, 11R*, 12R*, 16S*]]-7,1 1-dihydroxy-8,8,10,12,1 6-pentamethyl-3-[1-methyl-2-(2-methyl-4-thiazolyl)ethenyl]-4-aza-17-oxabicyclo[14.1.0]heptadecane-5,9-dione, and [1S-[1R*, 3R*(E), 7R*, 10S*, 11R*, 12R*, 16S*]]-3-[2-[2-(aminomethyl)-4-thiazolyl]-1-methylethenyl]-7, 11-dihydroxy-8,8,10,12,16-pentamethyl-4,17-dioxabicyclo[14.1.0]heptadecane-5,9-dione, the cyclin dependent kinase exemplified in U.S. Pat. No. 6,040,321; the famesyltransferase inhibitors exemplified in U.S. Pat. No. 6,011,029 as well as (R)-7-cyano-2,3,4,5-tetrahydro-1-(1H-imidazol-4-ylmethyl)-3-(phenylmethyl)-4-(2-thienylsulfonyl)-1H-1,4-benzodiazepine, mesylate salt.

The methods of the instant invention may employ pharmaceutical compositions which comprise at least one small molecule Eg5 protein inhibitor. Preferred compounds have formula I, IIA or IIIA, or the formula described in US Serial No: to be determined, filed Mar. 22, 2002, identified by attorney docket number LD 0300, and a pharmaceutically acceptable carrier and may additionally comprise at least one other antineoplastic agent. The compositions used in the methods of the present invention may further comprise one or more pharmaceutically acceptable additional ingredient(s) such as alum, stabilizers, antimicrobial agents, buffers, coloring agents, flavoring agents, and the like. The antineoplastic agents, small molecule Eg5 protein inhibitors, and compositions of the present invention may be administered orally or parenterally including the intravenous, intramuscular, intraperitoneal, subcutaneous, rectal and topical routes of administration.

For oral use, the antineoplastic agents, small molecule Eg5 protein inhibitors and compositions of this invention may be administered, for example, in the form of tablets or capsules, or as aqueous solutions or suspensions. In the case of tablets for oral use, carriers which are commonly used include lactose and corn starch, and lubricating agents such as magnesium stearate are commonly added. For oral administration in capsule form, useful carriers include lactose and corn starch. When aqueous suspensions are used for oral administration, emulsifying and/or suspending agents are commonly added. In addition, sweetening and/or flavoring agents may be added to the oral compositions. For intramuscular, intraperitoneal, subcutaneous and intravenous use, sterile solutions of the active ingredient(s) are usually employed, and the pH of the solutions should be suitably adjusted and buffered. For intravenous use, the total concentration of the solute(s) should be controlled in order to render the preparation isotonic.

The combinations of the present invention may also be used in conjunction with other well known therapies that are selected for their particular usefulness against the condition that is being treated. If formulated as a fixed dose, the active ingredients of the combination compositions of this invention are employed within effective dosage ranges. Alternatively, the antineoplastic agents and small molecules may be administered separately in the appropriate effective dosage ranges. In a preferred embodiment of the present invention, the antineoplatic agent is administered in the effective dosage range prior to administration of the compounds of the present invention in the effective dosage range.

The present invention encompasses a method for the treatment of cancer wherein at least one antineoplastic agent and at least one compound of the present invention is administered simultaneously or sequentially. Thus, while a pharmaceutical formulation comprising an antineoplastic agent and a compound of the present invention may be advantageous for administering the combination for one particular treatment, prior administration of the antineoplastic agent may be advantageous in another treatment. It is also understood that the instant combination of antineoplastic agent and small molecule Eg5 protein inhibitor may be used in conjunction with other methods of treating cancer (preferably cancerous tumors) including, but not limited to, radiation therapy and surgery.

Daily dosages for human administration of the antineoplastic agent, radiation therapy and small molecule Eg5 protein inhibitors will normally be determined by the prescribing physician with the dosages generally varying according to the age, weight, and response of the individual patient, as well as the severity of the patient's symptoms.

In order to facilitate a further understanding of the invention, the following assays and examples are presented primarily for the purpose of illustrating more specific details thereof. The scope of the invention should not be deemed limited by the examples, but encompass the entire subject matter defined the claims.

ASSAYS

The pharmacological properties of the Eg5 inhibitors of this invention may be confirmed by a number of pharmacological assays. The exemplified pharmacological assays which follow have been carried out with the compounds according to the invention and their salts. The compounds of examples 1 to 32 exhibited antiproliferative activity.

Cell Culture

Cell lines were maintained in RPMI-1640 plus 10% fetal bovine serum. Human cell lines used in one or more of the following assays described below included but were not limited to A2780 ovarian carcinoma, HCT1 16, colorectal carcinoma; HT-29, colorectal adenocarcinoma; SK-BR-3, mammary adenocarcinoma; Saos-2, osteosacroma; PC-3, prostate adenocarcinoma; and LX-1, lung carcinoma. The kangaroo rat kidney epitheilal cell line, PTK2, was also used.

72-Hour Proliferation Assay

Cells were plated at a density of about 3,000-6,000 cells/well, depending upon the cell line used, in a 96-well plate. The cultures were grown overnight. Cells were then treated in triplicate with a seven concentration dose-response curve. The maximum concentration of DMSO never exceeded 0.5%. Cells were exposed to compound for about 72 hours. Proliferation was measured using XTT or MTS from Promega. The compounds having formulae I and IIA exhibited activity in the 72-hour cell proliferation assay, inhibiting cell proliferation with at an IC₅₀ less than or equal to about 10 μM.

Clonogenic Growth Assay

Colony growth inhibition was measured using a standard clonogenic assay. Briefly, about 200 cells/well were seeded into 6-well tissue culture plates (Falcon, Franklin Lakes, N.J.) and allowed to attach for 18 hours. Assay medium consisted of RPMI-1640 plus 10% fetal bovine serum. Cells were then treated in duplicate with a six concentration dose-response curve. The maximum concentration of DMSO never exceeded 0.25%. Cells were exposed to compound for about 4 to 24 hours. Compound was then removed and the cells were washed with 2 volumes of PBS. The normal growth medium was then replaced. Colonies were fed with fresh media every third day. Colony number was scored on day 10-14 using a Optimax imaging station. The compound concentration required to inhibit 50% or 90% of colony formation (IC₅₀ or IC₉₀, respectively) was determined by non-linear regression analysis. When exposed to cells for about 24 hours, the compounds of the present invention exhibited activity in the clonogenecity assay.

Combination Studies—Clonogenic Growth Assays

Combination studies to examine the use of the Eg5 inhibitors of the present invention in combination with other antineoplastic agents were conducted essentially the same as the standard colony growth assay with the exception of compound treatment. In the combination studies, the cells were treated with both a compound of formula land another antineoplastic agent. The compounds were administered simultaneously or sequentially; both the order of sequence and length of treatment (about 1 to 24 hours) were varied. Data evaluation was based upon the isobologram analysis and the envelope of additivity, using the line of multiplicity which compares the survival fractions of combination treatments with those of single drug treatments.

Cell Cycle Analysis

The cell cycle profile of cells treated with compounds of the present invention was monitored by flow cytometry. Briefly, cells were seeded at a density of about 2×10⁵ per well in standard 6 well culture plates and permitted to grow for about 17 hours. Cells were then exposed to compounds of the present invention at varying concentrations for about 2 to 24 hours. Following exposure, cell populations were harvested, stained with propidium iodide to determine DNA content and also stained with the appropriate immunological reagent for protein biomarkers of mitosis and apoptosis, including, but not limited to, for example, anti-phospho-ThreonineProline, anti-M Phase Phospoprotein 2 (MMP2), and anti-p85 PARP. The compounds of the present invention exhibited activity in the cell cycle profile analysis assay, producing significant increases in the mitotic and apoptotic fractions of the total cell population.

Immunocytochemistry Assays

Cells were plated at a density of 200 to 2000 cells per well in 4 chamber glass slides and allowed to attach overnight. Cells were then treated with compounds of the present invention at concentrations of 100 nM to 50 μM for about 4 to 30 hours, fixed and permeabilized for subsequent staining. Stain reagents included, for example, propidium iodide, DAPI, rhodamine phalloidin, anti-αtubulin, anti-βtubulin, anti-γtubulin, and the appropriate fluorescent-tagged secondary antibodies. Cells were imaged by fluorescent and confocal fluorescent microscropy. The compounds of the present invention inhibited bipolar spindle formation and induced a monoastral array of microtubules.

### EXAMPLE 1

5-Cyano-3,6-dihydro-4-methyl-6-(3-nitrophenyl)-2-thioxo-1-(2H)-pyrimidinecarboxylic Acid, 1-ethyl Ester

A. Step 1

A mixture of 6.42 g of acetoacetamide, 8.0 g of 3-nitrobenzaldehyde, 0.61 ml of acetic acid, and 0.21 ml of piperidine in 30 ml of toluene was heated to reflux. A Dean Stark trap was used to azeotrope the water produced. After refluxed for 2 h, the reaction mixture was cooled to room temperature, with a lot of solid appeared, it was treated with a solution of 300 ml of EtOAc and 25 ml MeOH, the solid was then filtered off, rinsed with 15 ml of EtOAc twice to give 3.1 g of desired product in 25% yield.

B. Step 2

A mixture of 200 mg of the compound of Example 1, Step 1, 198 mg of 2-(4-methoxybenzyl)-2-thiopseudourea HCl salt, 84 mg of sodium acetate in 3.6 ml DMF was heated at 85° C. for 15 h, then cooled to room temperature. The resulting reaction mixture was purified by preparative HPLC using a (YMC S5 ODS 20×100 mm) column, the desired fraction was concentrated to dryness. Saturated NaHCO₃ (50 ml) was added and extracted with EtOAc (3×50 ml), combined EtOAc extracts were washed with 30 ml of brine, dried with MgSO₄, filtered and concentrated under vacuum to give 126.1 mg desired product in 36% yield.

C. Step 3

A mixture of the compound of Example 1, Step 2 (86.5 mg) and Burgess reagent (150 mg) in 7.0 ml of anhydrous THF was stirred at room temperature for 1 h, concentrated under vacuum, then purified by preparative HPLC using a YMC S5 (ODS 20×100 mm) column to give 80.8 mg of desired product in 87% yield.

D. Step 4

To a solution of the compound of Example 1, Step 3 (60 mg) and pyridine (0.1 ml) in 0.6 ml of CH₂CH₂, 17 μl of ethylchloroformate was added, after stirring for 2.5 h, another 22 μl of ethylchloroformate was added, the reaction mixture was stirred for 2 h, then 0.3 ml of trifluoroacetic acid was added, the resulting mixture was stirred for another 1 h, and concentrated under vacuum, diluted with DMF, MeOH and a little CH₂CH₂, filtered, then purified by preparative HPLC using a (YMC S5 ODS 20×100 mm) column to give 22.5 mg of product in 42.7% yield. MS (M−H)⁺=345. HPLC RT=2.85 min (YMC S5 ODS column 4.6×50 mm, 10-90% aqueous methanol over 4 minutes containing 0.2% phosphoric acid, 4ml/min, monitoring at 220 nm)

### EXAMPLE 2

5-Cyano-3,6-dihydro-4-methyl-6-(3-nitrophenyl)-2-oxo-1-(2H)-pyrimidinecarboxylic Acid, 1-ethyl Ester

A. Step 1

10.92 g of NaHCO₃ was added portionwise to a solution of 7.83 g of the compound of Example 1, Step 1 and 7.48g of o-methylisourea hydrogen sulfate in DMF (100 ml), there was gas evolved. The reaction mixture was stirred for 2 h, then heated at 65° C. overnight, cooled to room temperature, diluted with 800 ml of EtOAc, washed with water (2×100 ml) and brine (1×100 ml). The organic layer was dried MgSO₄, filtered and concentrated under vacuum. The resulting residue was triturated in EtOAc-CH₂Cl₂-hexane to give 5.48 g of desired product as solid (56%).

B. Step 2

A mixture of the compound of Example 2, Step 1 (209 mg) and Burgess reagent (274.5 mg) in CH₂CH₂ (5 ml) and THF (10 ml) was stirred overnight. The reaction mixture was concentrated under vacuum, diluted with 150 ml of EtOAc, then washed with saturated NaHCO₃ (2×30 ml) and brine (1×30 ml), dried with MgSO₄, concentrated under vacuum. The resulting residue was purified silica gel chromatography to give 136 mg (69.4%) of desired product.

C. Step 3

1.23 ml of pyridine was added to a solution of 2.075 g of the compound of Example 2, Step 2 in CH₂CH₂ (30 ml) under argon at 0° C., then 0.87 ml of ethyl chloroformate was added slowly. The reaction mixture was warmed to room temperature and stirred for 3 h, diluted with a mixture of saturated of NaHCO₃ (50 ml) and brine (50 ml), extracted with EtOAc three times, the combined layers were washed with brine and dried with MgSO₄, filtered and concentrated under vacuum, purified by silica gel chromatography to give 2.57 g (98%) of desired product.

D. Step 4

2.5 ml of TFA was added to a solution of 1.44 g of the compound of Example 2, Step 3 in CH₃CN (25 ml) and H₂O (2.5 ml), the reaction mixture was stirred for 2 h, a lot of white solid appeared. The solid was filtered off, rinsed with CH₃CN (3×20 ml) and hexane (2×20 ml), dried in air to give 860 mg (62.2%) desired product. The filtrate was concentrated under vacuum, the solid was recrystallized in CH₃CN to give another 320 mg (23.2%) of product. MS (M−H)⁺=329. HPLC RT=2.53 min (YMC S5 ODS column 4.6×50 mm, 10-90% aqueous methanol over 4 minutes containing 0.2% phosphoric acid, 4 ml/min, monitoring at 220 nm)

### EXAMPLE 3

5-Cyano-3,6-dihydro-4-methyl-6-(3-nitrophenyl)-2-oxo-1-(2H)-pyrimidinecarboxylic Acid, 1-ethyl Amide.

A. Step 1

To a solution of the compound of Example 2, Step 2 (100 mg; 0.37 mmol) and pyridine (0.74 mmol; 18 μL) in dichloroethane (40 μL) was added ethyl chloroformate (81 mg; 0.40 mmol) and the resulting solution was stirred at room temperature for 1.5 hours. The reaction mixture was diluted with saturated NaHCO₃ (30 μL), extracted with ethyl acetate (3×50 μL), dried (MgSO₄) and concentrated in vacuo to afford a white foam. Purification by chromatography (SiO₂: 20% EtOAc/hexane) afforded the desired compound as a colorless foam (99 mg; 62%)

B. Step 2

To a solution of the compound of Example 3, Step 1 (12 mg; 27 μmol) in THF (0.1 mL) was added 2M ethylamine in THF solution (15 μL; 30 mmol) in one portion at room temperature and the resulting yellow solution was stirred 30 minutes. Dilution of the reaction mixture with methanol (1.8 mL) afforded a yellow solid which was collected by suction filtration and purified by preparative HPLC to afford the title compound as a white solid (20 mg; 22%).

In contrast to the method of Example 2 above, in this case the 2-methoxy group hydrolyzed during isolation and purification to afford the dihydropyrimidinone ring without the need for treatment with TFA (Example 2, Step 4)

### EXAMPLE 4

5-Cyano-3,6-dihydro-4-methyl-6-(3-nitrophenyl)-2-oxo-1-(1-oxobutyl)-(2H)-pyrimidine

A. Step 1

23.7 μl of butyryl chloride was added to a solution of 52 mg of the compound of Example 2, Step 2 and 0.15 ml of pyridine in 0.6 ml of anhydrous CH₂CH₂, the reaction mixture was stirred for 1 h, then 24 μl of butyryl chloride was added, the reaction was stirred for 1.5 h, purified by preparative HPLC using a YMC S5 (ODS 20×100 mm) column to give 30 mg desired product.

B. Step 2

A solution of 30 mg of the compound of Example 4, Step 1, 0.2 ml of H₂O and 0.2 ml of TFA in 1.2 ml CH₃CN was stirred for 1.5 h, it was added another 0.1 ml of TFA and stirred for another 2.5 h. The reaction mixture was concentrated under vacuum, and purified by preparative HPLC using a YMC S5 (ODS 20×100 mm) column to give 11.8 mg desired product. MS (M-H)+=327. HPLC RT=3.06 min (YMC S5 ODS column 4.6×50 mm, 10-90% aqueous methanol over 4 minutes containing 0.2% phosphoric acid, 4 ml/min, monitoring at 220 nm)

### EXAMPLE 5

enantio 5-Cyano-3,6-dihydro-4-methyl-6-(3-nitrophenyl)-2-oxo-1-(2H)-pyrimidinecarboxylic Acid, 1-ethyl Ester (enantiomer A)

53 mg of the compound of Example 2, Step 4 was dissolved in absolute EtOH, preparative chiral separation was carried out using a Chiralcel OD-H S5 (4.6×250 mm) column, 20 mg of enantiomer A and 27 mg of enantiomer B were obtained. MS (M−H)⁺=329. HPLC-Chiral RT=10.44 min (Chiralcel OD-H, S5, column 4.6×250 mm, 10% MeOH/10% EtOH /Heptane, 1.0 ml /min, monitoring at 220 nm, 94.7% ee)

### EXAMPLE 6

enantio 5-Cyano-3,6-dihydro-4-methyl-6-(3-nitrophenyl)-2-oxo-1-(2H)-pyrimidinecarboxylic Acid, 1-ethyl Ester (enantiomer B)

MS (M−H)⁺=329. HPLC-Chiral RT=12.92 min (Chiralcel OD-H, S5, column 4.6×250 mm, 10% MeOH /10% EtOH /Heptane, 1.0 ml /min, monitoring at 220 nm, 99.64% ee)

### EXAMPLE 7

5-Cyano-3,6-dihydro-4-methyl-6-(3-aminophenyl)-2-oxo-1-(2H)-pyrimidinecarboxylic Acid, 1-ethyl Ester

A solution of 12 mg of the compound of Example 1, Step 4 in ethanol was treated with 100 mg of tin (II) chloride and heated to reflux under argon for 90 min., the reaction was cooled down and quenched with saturated NaHCO₃ solution and extracted with EtOAc (3×50 ml). The combined organic layer was washed with H₂O, dried with Na₂SO₄ and concentrated under vacuum. It was triturated with hexane and ether to give 8 mg of crude product, which was further purified by preparative HPLC to afford 3 mg of desired product as TFA salt. MS (M+H)⁺=301. HPLC RT=1.685 min (YMC S5 ODS column 4.6×50 mm, 10-90% aqueous methanol over 4 minutes containing 0.1% of TFA, 4 ml/min, monitoring at 220 nm)

### EXAMPLE 8

5-Cyano-3,6-dihydro-4-methyl-6-(3-(N,N-dimethyl)aminophenyl)-2-oxo-1-(2H)-pyrimidinecarboxylic Acid, 1-ethyl Ester

A solution of 12 mg of the compound of Example 7 in CH₃CN (1 ml) was added paraformaldehyde (40 mg), sodium cyanoborohydride (30 mg) followed by 2 drops of acetic acid. The reaction mixture was stirred at room temperature for 2 h, then quenched with saturated NaHCO₃ solution and extracted with EtOAc three times. The combined organic layer was washed with brine, dried with Na₂SO₄ and concentrated under vacuum. The resulting residue was purified by preparative HPLC to yield 3.2 mg of desired product as TFA salt. MS (M+H)⁺=329. HPLC RT=1.76 min (YMC S5 ODS column 4.6×50 mm, 10-90% aqueous methanol over 4 minutes containing 0.1% of TFA, 4 ml/min, monitoring at 220 nm) Examples 9 through 15 were prepared using the methods of Example 2 with the substitution of an appropriate benzaldehyde in Step 1.

### EXAMPLE 9

5-Cyano-3,6-dihydro-4-methyl-6-(3-trifluoromethylphenyl)-2-oxo-1-(2H)-pyrimidinecarboxylic Acid, 1-ethyl Ester

HPLC-HI 100% at 2.84 min (YMC S5 ODS column 4.6×50 mm, 10-90% aqueous methanol over 4 minutes containing 0.1% of TFA , 4 ml/min, monitoring at 220 nm). MS: [M+H]⁺=354.

### EXAMPLE 10

5-Cyano-3,6-dihydro-4-methyl-6-(2,3-dichlorophenyl)-2-oxo-1-(2H)-pyrimidinecarboxylic Acid, 1-ethyl Ester

HPLC-HI 100% at 3.2 min (YMC S5 ODS column 4.6×50 mm, 10-90% aqueous methanol over 4 minutes containing 0. 1% of TFA , 4 ml/min, monitoring at 220 nm). MS: [M−H]⁻=352.

### EXAMPLE 11

5-Cyano-3,6-dihydro-4-methyl-6-(3-methoxyphenyl)-2-oxo-1-(2H)-pyrimidinecarboxylic Acid, 1-ethyl Ester

HPLC-HI 100% at 2.42 min (YMC S5 ODS column 4.6×50 mm, 10-90% aqueous methanol over 4 minutes containing 0.1% of TFA , 4 ml/min, monitoring at 220 nm). MS: [M+H]⁺=316.

### EXAMPLE 12

5-Cyano-3,6-dihydro-4-methyl-6-(3,5-dichlorophenyl)-2-oxo-1-(2H1)-pyrimidinecarboxylic Acid, 1-ethyl Ester

HPLC-HI 87% at 3.26 min (YMC S5 ODS column 4.6×50 mm, 10-90% aqueous methanol over 4 minutes containing 0.1% of TFA , 4 ml/min, monitoring at 220 nm). MS: [M−HL]⁻=352.

### EXAMPLE 13

5-Cyano-3,6-dihydro-4-methyl-6-(3,4-dichlorophenyl)-2-oxo-1-(2H1)-pyrimidinecarboxylic Acid, 1-ethyl Ester

HPLC-HI 100% at 3.197 ml/min (YMC S5 ODS column 4.6×50 mm, 10-90% aqueous methanol over 4 minutes containing 0.1% of TFA , 4 ml/min, monitoring at 220 nm). MS: [M−H]⁻=352.

### EXAMPLE 14

5-Cyano-3,6-dihydro-4-methyl-6-(3-cyanophenyl)-2-oxo-1-(2H1)-pyrimidinecarboxylic Acid, 1-ethyl Ester

HPLC-HI 93% at 2.32 min (YMC S5 ODS column 4.6×50 mm, 10-90% aqueous methanol over 4 minutes containing 0.1% of TFA , 4 ml/min, monitoring at 220 nm). MS: [M+H]⁺=311.

### EXAMPLE 15

5-Cyano-3,6-dihydro-4-methyl-6-(4-methoxyphenyl)-2-oxo-1-(2H)-pyrimidinecarboxylic Acid, 1-ethyl Ester

HPLC-HI 100% at 2.55 min (YMC S5 ODS column 4.6×50 mm, 10-90% aqueous methanol over 4 minutes containing 0.1% of TFA , 4 ml/min, monitoring at 220 nm). MS: [M+H]⁺=316.

### EXAMPLE 16

5-Cyano-3,6-dihydro-4-methyl-6-(4-methylphenyl)-2-oxo-1-(2H)-pyrimidinecarboxylic Acid, 1-ethyl Ester

A. Step 1

A cloudy solution of 3-aminocrotononitrile (41 g, 0.5 mol) in Et₂O (500 ml) was added dropwise to the 15% HCl solution (115 ml) at 0° C. over 30 min with vigorous stirring, and the reaction mixture was stirred at 0° C. for 15 rain. The aqueous solution was then separated, extracted with Et₂O (2×125 ml), the combined organic phases dried with Na₂SO₄. Triethyl orthoformate (83 ml) in a 500 ml three-neck flask equipped with addition funnel and distillation set was stirred in 60° C.-65° C. oil bath, the above ether solution was added dropwise such that the rate of addition was equal to the rate of distillation. An additional 83 ml of triethyl orthoformate was added to the reaction when the addition of the ether solution was half complete, the oil bath temperature was slowly raised to 100° C., and the reaction mixture was then stirred for 5 h. Distillation gave 26.6 g (38%) of desired red solid product at 150-155° C./2 mm Hg.

B. Step 2

To a mixture of O-methylisourea sulfate (9.9 g, 80 mmol), the compound of Example 16, Step 1 (7.4 g, 53 mmol) and ethanol (90 ml) was added Et₃N (11 ml, 80 mmol). The mixture was stirred at room temperature for 15 min, then stirred at 66° C. for 3 h, and concentrated to remove EtOH. EtOAc (80 ml) and H₂O (80 ml) were added, the aqueous layer were separated and extracted with EtOAc (2×80 ml), the combined organic layer were dried with Na₂SO₄, concentrated to give brown solid, which was dissolved in EtOAc, filtered through a silica gel pad, washed with EtOAc/heptane (1/1) to remove dark color, and the combined filtrate was concentrated. The solid thus obtained was recrystallized in heptane EtOAc to give yellow crystal 5.18 g in 65% yield.

C. Step 3

A solution of p-tolylmagnesium bromide in ether (1M, 1 ml, 1 mmol) was added dropwise to a solution of the compound of Example 16, Step 2 (75 mg, 0.5 mmol) in THF (2 ml) at 0° C. under argon. The reaction mixture was stirred at the temperature for 1.5 h, another 3 ml of Grignard reagent was added at −78° C., the reaction was slowly warmed to room temperature and stirred for 2 min. Saturated NH₄Cl (5 ml) and H₂O (5 ml) were added, the mixture was extracted with EtOAc (2×15 ml), the combined organic layer was dried, concentrated and chromatographed on silica gel to give 45.6 mg of desired product in 91% yield.

D. Step 4

To a solution of the compound of Example 16, Step 3 (109 mg, 0.45 mmol) was added pyridine (0.2 ml, 2.5 mmol) in dry CH₂Cl₂ (5 ml) followed by ethyl chloroformate (0.1 ml, 1.05 mmol), and the resulting reaction mixture was stirred at room temperature overnight. MeOH was added, the resulting mixture was stirred for 15 min, concentrated, and chromatographed on silica gel column to give 100 mg desired product as colorless oil (71%).

E. Step 5

A mixture of the compound of Example 15, Step 4 (100 mg, 0.32 mmol), H20 (0.7 ml), CH₃CN (0.5 ml) and TFA (7 ml) was stirred at room temperature for 2 h. The solution was then concentrated to remove CH₃CN, saturated NaHCO₃ was added to make the mixture basic, the white solid precipitate was filtered, washed with H₂O, and dried to give the desired product (64 mg). The crude product was dried and recrystallized from EtOH/H₂O to give another 20 mg desired product as white solid. MS (M+H)⁺=300. HPLC RT=3.40 min (YMC S5 ODS column 4.6×50 mm, 10-90% aqueous methanol over 4 minutes containing 0.2% phosphoric acid, 4 ml/min, monitoring at 220 nm)

### EXAMPLE 17

5-Cyano-3,6-dihydro-4-methyl-6-cyclohexyl-2-oxo-1-(2H)-pyrimidinecarboxylic Acid, 1-ethyl Ester

To a solution of the compound of Example 16, Step 2 (30 mg, 0.2 mmol) in THF (1.2 ml) was added cyclohexylmagnesium chloride (2 M in ether, 1.0 ml, 2 mmol) at -44° C. under argon, the reaction was slowly warmed to room temperature, and stirred for 10 min. Saturated NH₄Cl was added, the resulting mixture was extracted several times with EtOAc, the combined organic layer dried, filtered through a silica gel pad, and concentrated to give yellow oil. The oil was dissolved in CH₂CH₂ (2 ml), then pyridine (80 μl, 0.9 mmol) and ethyl chloroformate (50 μl, 0.5 mmol) were added, the mixture was stirred at room temperature for 30 min, stirred for another 10 min after which H₂O (25 μl) and EtOAc were added, and the mixture dried over Na₂SO₄, filtered through a silica gel pad, and concentrated to give yellow oil. The oil was dissolved in CH₃CN (2 ml), H₂O (0.3 ml) and TFA (0.2 ml) were added, and the mixture stirred at room temperature for 2 h. Saturated NaHCO₃ solution and EtOAc were added, the aqueous layer was separated and extracted with EtOAc, and the combined organic layer was dried over Na₂SO₄, concentrated and chromatographed on silica gel to give 35 mg of desired product as yellow foam (60%). MS (M+H)⁺=392. HPLC RT=3.60 min (YMC S5 ODS column 4.6×50 mm, 10-90% aqueous methanol over 4 minutes containing 0.2% phosphoric acid, 4 ml/min, monitoring at 220 nm)

### Example 18

5-Cyano-3,6-dihydro-4-methyl-6-phenyl-2-oxo-1-(2H)-pyrimidinecarboxylic Acid, 1-ethyl Ester

To a solution of the compound of Example 16, Step 2 (55 mg, 0.37 mmol) in dry THF (2 ml) was added phenylmagnesium bromide (2 M in THF, 2 ml, 4 mmol) dropwise at −78° C. under argon. After addition, the reaction was slowly warmed to room temperature and stirred for about 10 min, until starting material disappeared. Saturated NH₄Cl solution and H₂O were added, the mixture was extracted with EtOAc for two times, and the combined organic layer was dried over Na₂SO₄, concentrated and chromatographed on silica gel to give solid intermediate. The solid was dissolved in CH₂Cl₂ (5 ml), pyridine (0.15 ml, 1.8 mmol) and ethyl chloroformate (0.1 ml, 1 mmol) were added, and the reaction mixture was stirred at room temperature for 0.5 h. The reaction was quenched with 50 μl of H₂O, diluted with 5 ml of EtOAc, the resulting mixture was dried over Na₂SO₄, filtered through silica gel column to give the intermediate as an oil. The oil was dissolved in CH₃CN (5 ml), H₂O (0.5 ml) and TFA (0.4 ml) were added, the reaction mixture stirred for 1.5 h, and concentrated in vacuo. Saturated NaHCO₃ solution was added to neutralize the mixture, and the precipitate was then filtered and air dried. Recrystallization in EtOAc/heptane to give 70 mg solid product in 66% yield. MS (M+H)⁺=286. HPLC RT=1.28 min. (Phenom-Prime S5 C18 4.6×30 mm, 10-90% aqueous methanol over 2 minutes containing 0.1% TFA, 5 ml/min, monitoring at 220 nm) Examples 19 through 24 were prepared using the method of Example 18 with the substitution of an appropriate arylmagnesiumhalide.

### EXAMPLE 19

5-Cyano-3,6-dihydro-4-methyl-6-(2-methylphenyl)-2-oxo-1-(2H)-pyrimidinecarboxylic Acid, 1-ethyl Ester

MS (M+H)⁺=300. HPLC RT=1.41 min. (Phenom-Prime S5 C18 4.6×30 mm, 10-90% aqueous methanol over 2 minutes containing 0.1% TFA, 5 ml/min, monitoring at 220 nm)

### EXAMPLE 20

5-Cyano-3,6-dihydro-4-methyl-6-(3-chlorophenyl)-2-oxo-1-(2H)-pyrimidinecarboxylic Acid, 1-ethyl Ester

MS (M+H)⁺=320. HPLC RT=1.43 min. (Phenom-Prime S5 C18 4.6×30 mm, 10-90% aqueous methanol over 2 minutes containing 0.1% TFA, 5 ml/min, monitoring at 220 nm)

### EXAMPLE 21

5-Cyano-3,6-dihydro-4-methyl-6-(3-fluorophenyl)-2-oxo-1-(2H)-pyrimidinecarboxylic Acid, 1-ethyl Ester

MS (M+H)⁺=304. HPLC RT=1.29 min. (Phenom-Prime S5 C18 4.6×30 mm, 10-90% aqueous methanol over 2 minutes containing 0.1% TFA, 5 ml/min, monitoring at 220 nm)

### EXAMPLE 22

5-Cyano-3,6-dihydro-4-methyl-6-(4-chlorophenyl)-2-oxo-1-(2H)-pyrimidinecarboxylic Acid, 1-ethyl Ester

MS (M+H)⁺=320. HPLC RT=1.44 min. (Phenom-Prime S5 C18 4.6×30 mm, 10-90% aqueous methanol over 2 minutes containing 0.1% TFA, 5 ml/min, monitoring at 220 nm)

### EXAMPLE 23

5-Cyano-3,6-dihydro-4-methyl-6-(4-fluorophenyl)-2-oxo-1-(2H)-pyrimidinecarboxylic Acid, 1-ethyl Ester

MS (M+H)⁺=304. HPLC RT=3.21 min (YMC S5 ODS column 4.6×50 mm, 10-90% aqueous methanol over 4 minutes containing 0.2% phosphoric acid, 4 ml/min, monitoring at 220 nm)

### EXAMPLE 24

5-Cyano-3,6-dihydro-4-methyl-6-(2-fluorophenyl)-2-oxo-1-(2H)-pyrinidinecarboxylic Acid, 1-ethyl Ester

MS (M+H)⁺=304. HPLC RT=3.05 min (YMC S5 ODS column 4.6×50 mm, 10-90% aqueous methanol over 4 minutes containing 0.2% phosphoric acid, 4 ml/min, monitoring at 220 nm)

### EXAMPLE 25

5-Cyano-2,6-dimethyl-4-(3-nitro-phenyl)-1,4-dihydro-pyridine-3-carboxylic Acid Ethyl Ester

A. 2-Acetyl-3-(3-nitro-phenyl)-acrylic Acid Ethyl Ester

A mixture of ethyl acetoacetate (8.6 g, 66 mmol), 3-nitrobenzaldehyde (10 g, 66 mmol), acetic acid (0.8 g), and pyridine (0.26 mL) in toluene (30 mL) was heated at reflux in a flask fitted with a Dean-Stark trap. After 2 hours 1.1 mL of water had been collected and the mixture was cooled and diluted with ethyl acetate, washed with water (100 mL), saturated sodium bicarbonate solution (100 mL), and brine (100 mL). The organic layer was collected, dried over sodium sulfate, filtered and concentrated. The residue was recrystallized from ether hexanes to yield a white solid (6 g, 35%).

B. 5-Cyano-2,6-dimethyl-4-(3-nitro-phenyl)-1,4-dihydro-pyridine-3-carboxylic Acid Ethyl Ester

A mixture of benzylidene from Step A (2.63 g, 10 mmol) and 3-aminocrotononitrile (0.86 g, 11 mmol) in ethanol (50 mL) was heated at reflux for 24 hours. The mixture was concentrated in vacuo and the residue was purified by silica gel chromatography eluting with hexanes/ethyl acetate (1:1) to yield the product as a solid (2.0 g, 61%). HPLC-HI 100% at 2.84 min (YMC S5 ODS column 4.6×50 mm, 10-90% aqueous methanol over 4 minutes containing 0.1% TFA, 4 mL/min, monitoring at 220 nm). MS: Found (M+H)⁺328. Calculated for C₁₇H₁₇N₃O₄: C 62.37; H 5.23; N 12.83. Found: C 62.08, H 5.18, N 12.67.

### EXAMPLE 26

5-Cyano-2,6-dimethyl-4-(4-nitro-phenyl)-1,4-dihydro-pyridine-3-carboxylic Acid Ethyl Ester

5-Cyano-2,6-dimethyl-4-(4-nitro-phenyl)-1,4-dihydro-pyridine-3-carboxylic acid ethyl ester, was synthesized according to the procedure for Example 25 with the only exception being the use of 4-nitrobenzaldehyde in Step A. HPLC-HI 93% at 2.89 min (YMC S5 ODS column 4.6×50 mm, 10-90% aqueous methanol over 4 minutes containing 0.1% TFA, 4 mL/min, monitoring at 220 nm). MS: Found (M+H)⁺328.

### EXAMPLE 27

5-Cyano-4-(3-cyano-phenyl)-2,6-dimethyl-1,4-dihydro-pyridine-3-carboxylic Acid Ethyl Ester

5-Cyano-4-(3-cyano-phenyl)-2,6-dimethyl-1,4-dihydro-pyridine-3-carboxylic acid ethyl ester, was synthesized according to the procedure for Example 25 with the only exception being the use of 3-cyanobenzaldehyde in Step A. HPLC-HI 83% at 2.71 min (YMC S5 ODS column 4.6×50 mm, 10-90% aqueous methanol over 4 minutes containing 0.1% TFA, 4 mL/min, monitoring at 220 nm). MS: Found (M+H)⁺308.

### EXAMPLE 28

5-Cyano-2,6-dimethyl-4-(3-trifluoromethyl-phenyl)-1,4-dihydro-pyridine-3-carboxylic Acid Ethyl Ester

5-Cyano-2,6-dimethyl-4-(3-trifluoromethyl-phenyl)-1,4-dihydro-pyridine-3-carboxylic acid ethyl ester, was synthesized according to the procedure for Example 25 with the only exception being the use of 3-trifluoromethylbenzaldehyde in Step A. HPLC-HI 95% at 3.21 min (YMC S5 ODS column 4.6×50 mm, 10-90% aqueous methanol over 4 minutes containing 0.1% TFA, 4 mL/min, monitoring at 220 nm). MS: Found (M+H)⁺351.

### EXAMPLE 29

5-Cyano-2,6-dimethyl-4-(3-methoxy-phenyl)-1,4-dihydro-pyridine-3-carboxylic Acid Ethyl Ester

5-Cyano-2,6-dimethyl-4-(3-methoxy-phenyl)-1,4-dihydro-pyridine-3-carboxylic acid ethyl ester, was synthesized according to the procedure for Example 25 with the only exception being the use of 3-methoxybenzaldehyde in Step A. HPLC-HI 97% at 2.81 min (YMC S5 ODS column 4.6×50 mm, 10-90% aqueous methanol over 4 minutes containing 0.1% TFA, 4 mL/min, monitoring at 220 nm). MS: Found (M+H)⁺313.

### EXAMPLE 30

5-Cyano-2,6-dimethyl-4-(3-nitro-phenyl)-1,4-dihydro-pyridine-3-carboxylic Acid Isopropyl Ester

5-Cyano-2,6-dimethyl-4-(3-nitro-phenyl)-1,4-dihydro-pyridine-3-carboxylic acid isopropyl ester, was synthesized according to the procedure for Example 25 with the only exception being the use of isopropyl acetoacetate in Step A. HPLC-HI 82% at 3.04 min (YMC S5 ODS column 4.6×50 mm, 10-90% aqueous methanol over 4 minutes containing 0.1% TFA, 4 mL/min, monitoring at 220 nm). MS: Found (M+H)⁺342.

### EXAMPLE 31

5-Cyano-4-(3,4-dichloro-phenyl)-2,6-dimethyl-1,4-dihydro-pyridine-3-carboxylic Acid Ethyl Ester

A. 2-Acetyl-3-(3,4-dichloro-phenyl)-acrylic Acid Ethyl Ester

A mixture of ethyl acetoactate (1.3 g, 10 mmol), 3,4-dichlorobenzaldehyde (1.8 g, 10 mmol), and BiCI₃ (315 mg) was heated at 80° C. for 3 hours. After cooling the mixture was diluted with ethyl acetate (100 mL) and washed with 10% sodium bicarbonate solution, 10% sodium hydrogensulfate, and brine. The organic layer was collected, dried over sodium sulfate, and concentrated. The resulting benzylidene product was used crude in Step B.

B. 5-Cyano-4-(3,4-dichloro-phenyl)-2,6-dimethyl-1,4-dihydro-pyridine-3-carboxylic Acid Ethyl Ester

A mixture of benzylidene from Step A (0.13 g, 0.4 mmol) and 3-aminocrotononitrile (44 mg, 0.5 mmol) in ethanol (1 ML) was heated at reflux for 24 hours. The mixture was concentrated in vacuo and the residue was purified by silica gel chromatography eluting with hexanes/ethyl acetate (7:3) to yield the product as a solid (15 mg, 11%). HPLC-HI 95% at 3.64 min (YMC S5 ODS column 4.6×50 mm, 10-90% aqueous methanol over 4 minutes containing 0.1% TFA, 4 mL/min, monitoring at 220 nm). MS: Found (M+H)⁺352.

### EXAMPLE 32

5-Cyano-4-(3,5-dichloro-phenyl)-2,6-dimethyl-1,4-dihydro-pyridine-3-carboxylic Acid Ethyl Ester

5-Cyano-4-(3 ,5-dichloro-phenyl)-2,6-dimethyl-1 ,4-dihydro-pyridine-3-carboxylic acid ethyl ester, was synthesized according to the procedure for Example 31 with the only exception being the use of 3,5-dichlorobenzaldehyde in Step A. HPLC-HI 90% at 3.50 min (YMC S5 ODS column 4.6×50 mm, 10-90% aqueous methanol over 4 minutes containing 0.1% TFA, 4 mL/min, monitoring at 220 nm). MS: Found (M+H)⁺352.

What is claimed is:

1. A method for treating a condition via modulation of Eg5 protein activity comprising administering to a mammalian species in need of such treatment an effective amount of at least one small molecule Eg5 protein inhibitor.

2. The method of claim 1 wherein said small molecule induces mitotic arrest and apoptosis.

3. The method of claim 1 wherein said small molecule has an IC₅₀ value of less than about 10 uM in a cell proliferation assay.

4. The method of claim 1 further comprising administering to said mammalian species at least one other anti-cancer agent in combination with said small molecule.

5. The method according to claim 1 wherein the condition is cancer.

6. The method according to claim 1 wherein said small molecule is a compound of formula I or IIA: 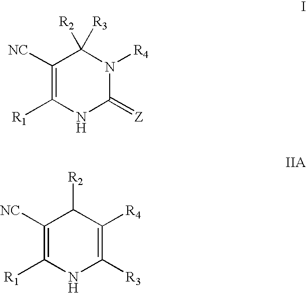
or an enantiomer, diastereomer, pharmaceutically acceptable salt, prodrug or solvate thereof, wherein
R₁ is selected from the group consisting of hydrogen, alkyl and cycloalkyl;
R₂ and R₃ are each independently selected from the group consisting of H, alkyl, aryl, heteroaryl, arylalkyl, cycloalkyl, cycloalkylalkyl, heterocycloalkylalkyl and heteroarylalkyl; R₂ and R₃ may also be taken together to form a carbocyclic or heterocyclic ring;
R₄ is selected from the group consisting of alkyl, arylalkyl, heterocycloalkyl, aminoalkyl, cycloalkylalkyl, heteroarylalkyl, heterocycloalkylalkyl, CN, COR₅, CO₂R₅ and CONR₅R₆;
R₅ and R6 are each independently selected from the group consisting of H, alkyl, arylalkyl, cycloalkylalkyl, heteroarylalkyl and heterocycloalkylalkyl; Z is selected from the group consisting of O, S and NR₈;
R₈ is selected from the group consisting of H, CN, sulfonamido, OR₇, alkyl, cycloalkyl, aryl, arylalkyl, heterocyclyl, heteroaryl and heteroarylalkyl; and R₇ is selected from the group consisting of H, alkyl, arylalkyl, cycloalkylalkyl, heterocycloalkylalkyl and heteroarylalkyl.

7. The method according to claim 6 wherein R₄ is selected from the group consisting of alkyl, arylalkyl, CO₂R₅, and CONR₅R₆.

8. The method according to claim 6 wherein R₄ is CO₂R₅ and Z is O.

9. The method according to claim 6 wherein R₄ is CONR₅R₆ and Z is O.

10. The method according to claim 6 wherein R₄ is selected from the group consisting of alkyl and arylalkyl; and Z is O.

11. The method according to claim 6 wherein R₁ is CH₃; R₂ is aryl; R₄ is CO₂R₅; R₅ is alkyl; and Z is O.

12. The method according to claim 6 wherein R₁ is CH₃; R₂ is aryl; R₄ is CONR₅R₆; R₅ is alkyl; and Z is O.
